(19) United States
(12) Patent Application Publication
(10) Pub. No.: US 2026/0354729 A1
(43) Pub. Date: Nov. 12, 2026

(54) SYNCHRONOUS ELECTROCARDIOGRAMS CREATED APPARATUS USING TWO LEAD ASYNCHRONOUS ELECTROCARDIOGRAMS

(71) Applicant: Medical AI Co., Ltd., Seoul (KR)

(72) Inventors: Joon Myoung Kwon, Seoul (KR); Yong-Yeon Jo, Seoul (KR)

(73) Assignee: Medical AI Co., Ltd., Seoul (KR)

(21) Appl. No.: 19/661,499

(22) Filed: Apr. 28, 2026

Related U.S. Application Data
(63) Continuation of application No. 18/558,526, filed on Aug. 17, 2022, now Pat. No. 12,635,926.

(30) Foreign Application Priority Data
Aug. 17, 2021 (KR) 10-2021-0107772

(51) Int. Cl. A61B 5/327; A61B 5/00; G16H 50/20

(52) U.S. Cl. CPC A61B 5/327; A61B 5/0006; A61B 5/002; G16H 50/20

(57) ABSTRACT

The present invention relates to a method for generating synchronous electrocardiograms based on two lead asynchronous electrocardiograms. According to the present invention, there is provided a method of generating synchronous electrocardiograms, the method including: collecting electrocardiogram data of a plurality of patients from a server operated by a hospital, and classifying the collected electrocardiogram data by style; constructing a deep learning algorithm, and training a constructed machine learning model on styles of electrocardiograms by inputting the classified electrocardiogram data into the constructed machine learning model; acquiring two lead electrocardiograms, measured at different times, using electrodes installed on the body of a measurement subject; and generating a plurality of virtual synchronous lead electrocardiograms by inputting the lead electrocardiograms of the measurement subject to the trained machine learning model.

### CROSS-REFERENCE TO RELATED APPLICATIONS

This application is a continuation of U.S. patent application Ser. No. 18/558,526, filed Nov. 1, 2023, which is a national stage entry of International Application No. PCT/KR2022/012277, filed Aug. 17, 2022, which is a continuation of Korean Patent Application No. 10-2021-0107772, filed Aug. 17, 2021. The content of each of the foregoing applications is incorporated herein in its entirety by reference.

### TECHNICAL FIELD

The present invention relates to a method for generating synchronous electrocardiograms based on two lead asynchronous electrocardiograms, and more specifically to a method for generating synchronous electrocardiogram information from two pieces of lead asynchronous electrocardiogram information, measured at different times, using a deep learning algorithm.

### BACKGROUND ART

An electrocardiogram is a graphical record of electrical potentials related to heartbeat on the surface of the human body. Such electrocardiograms include not only standard 12-lead electrocardiograms but also exercise stress electrocardiograms and Holter electrocardiograms. Electrocardiograms are used for the examination and diagnosis of circulatory diseases, and have the advantages of being simple, relatively inexpensive, non-invasive, and easily recorded repeatedly.

As for a standard 12-lead electrocardiogram used in hospitals, six electrodes are attached to the front of the chest, three electrodes (four electrodes when a ground electrode is included) are attached to the limbs, 12-lead information is all collected and combined, and then a disease is diagnosed. A 12-lead electrocardiogram records electrical potentials of the heart in 12 electrical directions centered on the heart. Through this, the disease of the heart confined to one area can be accurately diagnosed.

However, it is difficult to measure a 12-lead electrocardiogram at home or in everyday life because the chest must be exposed to attach chest electrodes to take a 12-lead electrocardiogram and it is difficult for the general public to attach nine electrodes (three limb electrodes and six chest electrodes) to correct locations. In addition, it is difficult to use a 12-lead electrocardiogram for real-time monitoring because it is difficult to move after the attachment of ten electrodes.

Recently, there has been proposed a technology for measuring the side leads of a plurality of electrocardiograms using a 1-lead electrocardiogram device such as a smart watch. However, when electrocardiograms are measured using the 1-lead electrocardiogram device, a problem arises in that the accurate state of the heart cannot be determined because the electrocardiograms are not measured at the same time.

A background technology of the present invention is disclosed in Korean Patent Application Publication No. 10-2015-0044311 (published on Apr. 24, 2015).

### DISCLOSURE

### Technical Problem

As described above, the present invention is intended to provide a method for generating synchronous electrocardiogram information from two pieces of lead asynchronous electrocardiogram information, measured at different times, using a deep learning algorithm.

### Technical Solution

According to an embodiment of the present invention for solving the above technical problem, there is provided a method of generating synchronous electrocardiograms using a synchronous electrocardiogram generation apparatus, the method including: collecting electrocardiogram data of a plurality of patients from a server operated by a hospital, and classifying the collected electrocardiogram data by style; constructing a deep learning algorithm, and training a constructed machine learning model on styles of electrocardiograms by inputting the classified electrocardiogram data into the constructed machine learning model; acquiring two lead electrocardiograms, measured at different times, using electrodes installed on the body of a measurement subject; and generating a plurality of virtual synchronous lead electrocardiograms by inputting the lead electrocardiograms of the measurement subject to the trained machine learning model.

Classifying the collected electrocardiogram data by style may include classifying a plurality of the collected electrocardiogram data by at least one style among gender, age, disease status, attachment positions of electrodes, waveform characteristics, waveform curves, and noise signals of each patient.

Training a constructed machine learning model on styles of electrocardiograms may include training the machine learning model on the correlations between a plurality of electrocardiogram data and electrocardiogram styles by inputting the plurality of electrocardiogram data and the electrocardiogram styles to the machine learning model.

Training a constructed machine learning model on styles of electrocardiograms may include constructing 12 machine learning models to perform training on styles for 12 standard electrocardiograms.

The 12 machine learning models may be constructed using one principle selected between an autoencoder and a generative adversarial network, or may be constructed using a mixture of the two principles.

Generating a plurality of virtual synchronous lead electrocardiograms may include generating 12 pieces of electrocardiogram data changed into each style by inputting the lead electrocardiograms of the measurement subject into the 12 machine learning models.

### Advantageous Effects

As described above, according to the present invention, synchronous electrocardiogram information is generated from two pieces of lead asynchronous electrocardiogram information using a deep learning algorithm, so that there can be provided highly accurate electrocardiograms that are not influenced by the age, gender, disease status, and attachment positions of electrodes of a measurement subject.

### DESCRIPTION OF DRAWINGS

FIG. 1 is a block diagram of a synchronous electrocardiogram generation apparatus according to an embodiment of the present invention;

FIG. 2 is a flowchart illustrating a method for generating synchronous electrocardiograms using a synchronous electrocardiogram generation apparatus according to an embodiment of the present invention;

FIG. 3 is an example diagram illustrating a machine learning model constructed based on the principle of an autoencoder;

FIG. 4 is an example diagram illustrating a machine learning model constructed based on the principle of a generative adversarial network;

FIG. 5 is an example diagram illustrating step S240 shown in FIG. 2; and

FIG. 6 is an example diagram illustrating electrocardiogram data generated in step S240 shown in FIG. 2.

### MODE FOR INVENTION

Preferred embodiments according to the present invention will be described in detail below with reference to the accompanying drawings. In this process, the thicknesses of lines or sizes of components shown in the drawings may be shown exaggerated for clarity and convenience of description.

Additionally, the terms to be described below are terms defined by taking into consideration the functions thereof in the present invention, and may vary depending on the intention or custom of a user or operator. Accordingly, the definitions of these terms should be made based on the content throughout the present specification.

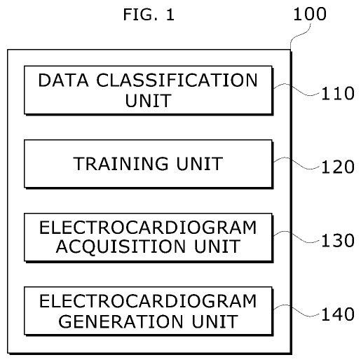

A synchronous electrocardiogram generation apparatus according to an embodiment of the present invention will be described in more detail below using FIG. 1.

FIG. 1 is a block diagram of a synchronous electrocardiogram generation apparatus according to an embodiment of the present invention.

As shown in FIG. 1, a synchronous electrocardiogram generation apparatus 100 according to an embodiment of the present invention includes a data classification unit 110, a training unit 120, an electrocardiogram acquisition unit 130, and an electrocardiogram generation unit 140.

The data classification unit 110 collects standard 12-lead electrocardiogram data from a hospital server, and classifies the collected electrocardiogram data by style.

The training unit 120 constructs a machine learning model, and trains the machine learning model to generate synchronous electrocardiograms by inputting a plurality of pieces of classified electrocardiogram data to the constructed machine learning model.

The electrocardiogram acquisition unit 130 acquires one or two pieces of lead electrocardiogram data from a measurement subject.

Finally, the electrocardiogram generation unit 140 generates a plurality of synchronous electrocardiograms by inputting the one or two pieces of lead electrocardiogram data to the trained machine learning model.

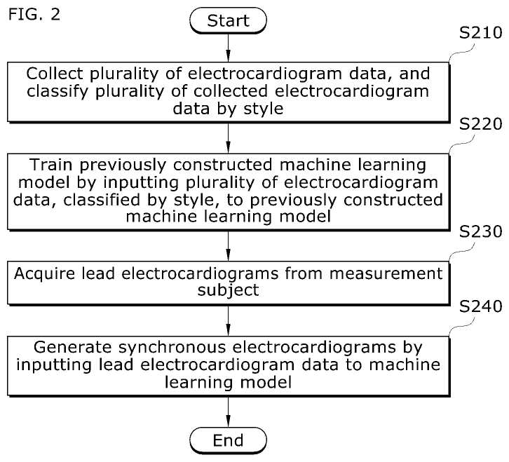

A method for generating synchronous electrocardiograms using the synchronous electrocardiogram generation apparatus 100 according to an embodiment of the present invention will be described in more detail below using FIGS. 2 to 6.

FIG. 2 is a flowchart illustrating a method for generating synchronous electrocardiograms using a synchronous electrocardiogram generation apparatus according to an embodiment of the present invention.

As shown in FIG. 2, the synchronous electrocardiogram generation apparatus 100 according to an embodiment of the present invention collects a plurality of pieces of electrocardiogram data from a hospital server, and classifies the plurality of pieces of collected electrocardiogram data by style in step S210.

More specifically, the data classification unit 110 collects standard 12-lead electrocardiograms. In this case, the standard 12-lead electrocardiograms refer to the electrocardiograms measured from electrodes attached to the human bodies of normal people and heart disease patients.

The data classification unit 110 classifies the plurality of collected standard 12-lead electrocardiograms by style. In this case, the style includes at least one of the gender, age, disease status, attachment positions of electrodes, waveform characteristics, waveform curves, and noise signals of each patient.

In more detail, each lead electrocardiogram has its own style. This includes not only the characteristics of the P, Q, R, S, or T waveform specified by existing medical knowledge, but also parts such as curves or noise signals that cannot be specified by existing medical knowledge.

Furthermore, the style of each lead electrocardiogram is influenced by the characteristics of a measurement subject. For example, the age and gender of a measurement subject influences the style of each lead electrocardiogram. In the case of the elderly, the heart muscle decreases, so that there is a tendency for the amplitude of the electrocardiogram to decrease. In the case of women, due to the breasts, the positions of electrocardiogram electrodes for measurement may be lowered or the distances between the heart and the electrodes may increase, causing deformation in the shape of an electrocardiogram.

Additionally, the style of each lead electrocardiogram is influenced by the disease of a measured person. In the case of chronic lung disease (chronic obstructive pulmonary disease), as the capacity of the lungs increases, the heart between the lungs is erected vertically, causing the electrical flow of the heart to change vertically in three-dimensional space.

Furthermore, the style of each lead electrocardiogram is also influenced by the attachment location of a corresponding electrode. For example, in the case of an I-lead electrocardiogram for which electrodes are attached to both arms and the potential difference between the two electrodes is used, there may be a difference between the case of performing measurement by attaching electrodes to both wrists and the case of performing measurement by attaching electrodes to both shoulders.

Therefore, in an embodiment of the present invention, a plurality of pieces of electrocardiogram data are classified by style.

When step S210 is completed, the training unit 120 trains a previously constructed machine learning model by inputting the plurality of pieces of electrocardiogram data, classified by style, to the previously constructed machine learning model in step S220.

First, the training unit 120 constructs a machine learning model. In this case, a plurality of machine learning models may be constructed according to the types of lead electrocardiograms. That is, the training unit 120 constructs 12 machine learning models. For example, in order to generate a V1-lead electrocardiogram, the training unit 120 constructs a machine learning model that learns a V1-lead style based on a large amount of V1-lead electrocardiogram data.

Meanwhile, machine learning models may be constructed using a principle selected between an autoencoder and a generative adversarial network, or may be constructed using a mixture of the two principles.

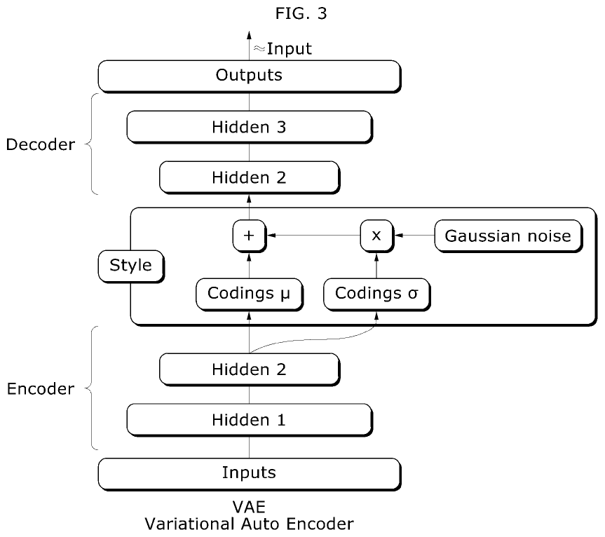

FIG. 3 is an example diagram illustrating a machine learning model constructed based on the principle of an autoencoder.

As shown in FIG. 3, the machine learning model constructed on the autoencoder principle includes an encoder configured to express the potential characteristics of data and a decoder configured to restore corresponding expressions to original data.

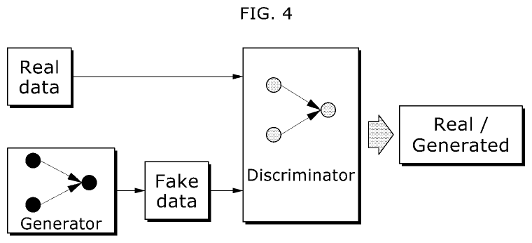

FIG. 4 is an example diagram illustrating a machine learning model constructed based on the principle of a generative adversarial network.

As shown in FIG. 4, the machine learning model constructed based on the principle of a generative adversarial network includes a generator and a discriminator. The generator generates synthetic electrocardiogram data by inputting randomly generated variables, and the discriminator classifies synthetic electrocardiogram data and real data as generated or real.

However, a basic generative adversarial network has a problem in that it is difficult to generate a specific lead electrocardiogram. Accordingly, in an embodiment of the present invention, the generator is changed into an autoencoder type. In other words, a machine learning model according to an embodiment of the present invention may be constructed using a mixture of the principles of a generative adversarial network and an autoencoder.

After the machine learning model has been constructed, the training unit 120 trains the machine learning model on the correlations between collected electrocardiogram data and corresponding styles by inputting the electrocardiogram data and the electrocardiogram styles to the machine learning model.

In the state in which step S220 is completed, the electrocardiogram acquisition unit 130 acquires lead electrocardiograms from the measurement subject in step S230.

More specifically, a measurement subject measures electrocardiograms through a 1-lead electrocardiogram device (not shown) such as a smart watch. That is, the electrocardiogram acquisition unit 130 acquires one or two pieces of lead electrocardiogram data from the 1-lead electrocardiogram device.

Thereafter, the electrocardiogram generation unit 140 generates synchronous electrocardiograms by inputting the acquired one or two pieces of lead electrocardiogram data to the trained machine learning model in step S240.

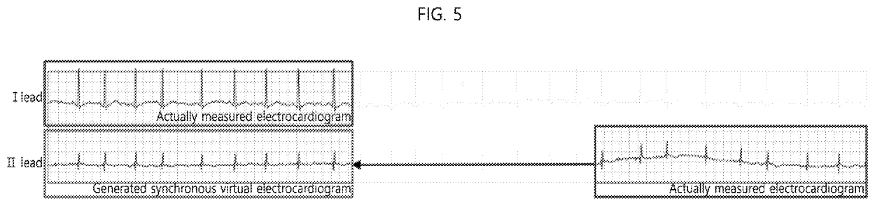

FIG. 5 is an example diagram illustrating step S240 shown in FIG. 2.

As shown in FIG. 5, it is assumed that the electrocardiogram acquisition unit 130 has acquired a V1-lead electrocardiogram. Then, the electrocardiogram generation unit 140 generates a V2 style electrocardiogram from the input lead electrocardiogram by inputting the V1-lead electrocardiogram to the machine learning model trained using V2-lead electrocardiogram data.

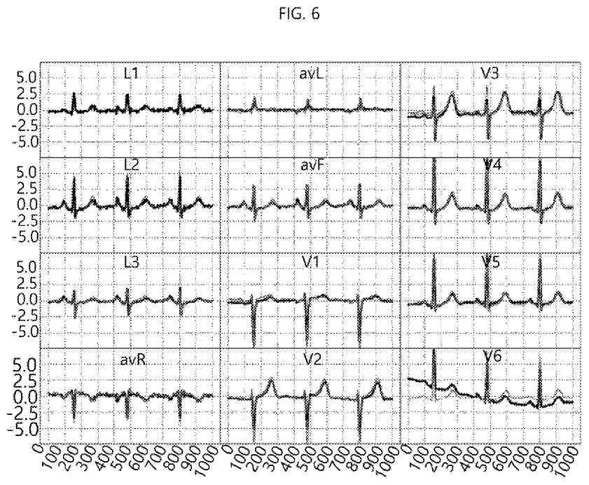

FIG. 6 is an example diagram illustrating electrocardiogram data generated in step S240 shown in FIG. 2.

As shown in FIG. 6, the thick lines denote actual electrocardiograms, and the thin lines denote the results obtained by generating electrocardiograms based on styles.

As described above, the synchronous electrocardiogram generation apparatus 100 according to an embodiment of the present invention identifies the style of the electrocardiogram, determines which type of electrocardiogram the currently input electrocardiogram data is, and generates a plurality of virtual synchronous lead electrocardiograms expected to have been measured at the same time synchronized according to each style.

As described above, according to the present invention, synchronous electrocardiogram information is generated from two pieces of lead asynchronous electrocardiogram information using a deep learning algorithm, so that there can be provided highly accurate electrocardiograms that are not influenced by the age, gender, disease status, and attachment locations of electrodes of a measurement subject.

While the present invention has been described with reference to the embodiments shown in the drawings, these are merely examples. It will be understood by those skilled in the art that various modifications and other equivalent embodiments may be made therefrom. Therefore, the true technical protection range of the present invention should be determined by the technical spirit of the following patent claims.

1-6. (canceled)

7. A method of generating synchronous electrocardiograms, the method comprising:
obtaining electrocardiogram data associated with a plurality of patients and classifying the electrocardiogram data by type of electrocardiograms to obtain classified electrocardiogram data, wherein a type comprises one or more ECG-specific characteristics or patient-specific characteristics that affect a signal of a corresponding electrocardiogram;
training a machine learning model on types of electrocardiograms using the classified electrocardiogram data to obtain a trained machine learning model;
acquiring at least one lead electrocardiogram using one or more electrodes on a body of a subject; and
generating one or more virtual synchronous lead electrocardiograms by inputting the at least one lead electrocardiogram of the subject into the trained machine learning model.

8. The method of claim 7, wherein classifying the electrocardiogram data by type comprises classifying the electrocardiogram data as at least one type among gender, age, disease status, attachment positions of electrodes, waveform characteristics, waveform curves, and noise signals of each patient.

9. The method of claim 8, wherein training the machine learning model on the types of electrocardiograms comprises training the machine learning model on correlations between a plurality of electrocardiogram data and electrocardiogram types by inputting the plurality of electrocardiogram data and the electrocardiogram types to the machine learning model.

10. The method of claim 9, wherein training the machine learning model on the types of electrocardiograms comprises constructing a plurality of machine learning models to perform training on types for 12 standard electrocardiograms.

11. The method of claim 10, wherein the plurality of machine learning models is constructed using one or both of an autoencoder or a generative adversarial network.

12. The method of claim 10, wherein generating the one or more virtual synchronous lead electrocardiograms comprises generating 12 pieces of electrocardiogram data modified into each type by inputting the electrocardiograms of the subject into the plurality of machine learning models.

13. A system for generating synchronous electrocardiograms, the system comprising:
one or more processors; and
one or more memory devices configured to store instructions that when executed by the one or more processors perform operations comprising:
obtaining electrocardiogram data associated with a plurality of patients and classifying the electrocardiogram data by type of electrocardiograms to obtain classified electrocardiogram data, wherein a type comprises one or more ECG-specific characteristics or patient-specific characteristics that affect a signal of a corresponding electrocardiogram;
training a machine learning model on types of electrocardiograms using the classified electrocardiogram data to obtain a trained machine learning model;
acquiring at least one lead electrocardiogram using one or more electrodes on a body of a subject; and
generating one or more virtual synchronous lead electrocardiograms by inputting the at least one lead electrocardiogram of the subject into the trained machine learning model.

14. The system of claim 13, wherein classifying the electrocardiogram data by type comprises classifying the electrocardiogram data as at least one type among gender, age, disease status, attachment positions of electrodes, waveform characteristics, waveform curves, and noise signals of each patient.

15. The system of claim 13, wherein training the machine learning model on the types of electrocardiograms comprises training the machine learning model on correlations between a plurality of electrocardiogram data and electrocardiogram types by inputting the plurality of electrocardiogram data and the electrocardiogram types to the machine learning model.

16. The system of claim 13, wherein training the machine learning model on the types of electrocardiograms comprises constructing a plurality of machine learning models to perform training on types for 12 standard electrocardiograms.

17. The system of claim 16, wherein the plurality of machine learning models is constructed using one or both of an autoencoder or a generative adversarial network.

18. The system of claim 16, wherein generating the one or more virtual synchronous lead electrocardiograms comprises generating 12 pieces of electrocardiogram data modified into each type by inputting the electrocardiograms of the subject into the plurality of machine learning models.

19. The system of claim 18, wherein the plurality of machine learning models comprises 12 machine learning models.

20. One or more non-transitory, computer-readable media storing instructions thereon that cause one or more processors to perform operations comprising:
obtaining electrocardiogram data associated with a plurality of patients and classifying the electrocardiogram data by type of electrocardiograms to obtain classified electrocardiogram data, wherein a type comprises one or more ECG-specific characteristics or patient-specific characteristics that affect a signal of a corresponding electrocardiogram;
training a machine learning model on types of electrocardiograms using the classified electrocardiogram data to obtain a trained machine learning model;
acquiring at least one lead electrocardiogram using one or more electrodes on a body of a subject; and
generating one or more virtual synchronous lead electrocardiograms by inputting the at least one lead electrocardiogram of the subject into the trained machine learning model.

21. The one or more non-transitory, computer-readable media of claim 20, wherein classifying the electrocardiogram data by type comprises classifying the electrocardiogram data as at least one type among gender, age, disease status, attachment positions of electrodes, waveform characteristics, waveform curves, and noise signals of each patient.

22. The one or more non-transitory, computer-readable media of claim 20, wherein training the machine learning model on types of electrocardiograms comprises training the machine learning model on correlations between a plurality of electrocardiogram data and electrocardiogram types by inputting the plurality of electrocardiogram data and the electrocardiogram types to the machine learning model.

23. The one or more non-transitory, computer-readable media of claim 20, wherein training the machine learning model on the types of electrocardiograms comprises constructing a plurality of machine learning models to perform training on types for 12 standard electrocardiograms.

24. The one or more non-transitory, computer-readable media of claim 23, wherein the plurality of machine learning models is constructed using one or both of an autoencoder or a generative adversarial network.

25. The one or more non-transitory, computer-readable media of claim 23, wherein generating the one or more virtual synchronous lead electrocardiograms comprises generating 12 pieces of electrocardiogram data modified into each type by inputting the electrocardiograms of the subject into the plurality of machine learning models.

26. The one or more non-transitory, computer-readable media of claim 25, wherein the plurality of machine learning models comprises 12 machine learning models.
